(19) United States
(12) Patent Application Publication
(10) Pub. No.: US 2025/0188011 A1
(43) Pub. Date: Jun. 12, 2025

(54) A PROCESS FOR THE RECOVERY AND SEPARATION OF FATTY ACIDS

(71) Applicant: Council of Scientific and Industrial Research, New Delhi (IN)

(72) Inventors: Amol Arvindrao KULKARNI, Pune (IN); Ranjit Shabu ATAPALKR, Pune (IN)

(21) Appl. No.: 18/844,238

(22) PCT Filed: Mar. 7, 2023
(86) PCT No.: PCT/IN2023/050210

§ 371 Date: Sep. 5, 2024

(30) Foreign Application Priority Data
Mar. 7, 2022 (IN) 202211012419

(51) Int. Cl. C07C 51/43

(52) U.S. Cl. CPC C07C 51/43

(57) ABSTRACT

A zero discharge, low temperature process of recovery and separation of the mixtures of mono and di acids is disclosed. The process provides recovery and separation of mixtures of mono and di acids (fatty acid) the acids with high yield and purity of products in substantially less period of time.

### FIELD OF THE INVENTION

The invention relates to a novel process for the recovery and separation of fatty acid. More particularly, the present invention relates to a process for the separation and recovery of acids synthesized by the oxidation of fatty acids oxidized using oxygen or ozone wherein the yields and purity of the product is highly enhanced.

### BACKGROUND AND PRIOR ART OF THE INVENTION

Fatty acids are oxidized employing harsh oxidizing agent such as H₂O₂ and other agents to yield mixture of acids in poor yields of 60-80% and with less purity. However, for cosmetic applications as in the case of Azelaic acid, highly pure product is desired. Conventionally this mixture of acids is separated by steam distillation or solvent extraction method at high temperature. But in large scale operations involving very large quantities of solvents, significant limitations on steam distillation are encountered. In large scale operations, high temperature processes are also not very desirable.

One of article (Molecules 2020, 25(8), 1882; https://doi.org/10.3390/molecules25081882) by Brenna et al., reports separation of a mixture of azelaic and pelargonic acids by repartition of the acids mixture between ethyl acetate and hot water (50° C. for 1 h in a 1:1 mixture of EtOAc and water) to afford an aqueous phase from which azelaic acid crystallised upon cooling. After three extraction cycles, azelaic acid could be recovered as a pure compound (>99% pure) in 73% yield. Pelargonic acid was isolated from the organic phase at 77% isolation yield, with 91% purity. The method needs cooling crystallization and is energy intensive.

One of patent document GB813842A discloses two-stage oxidation of oleic acid using molecular oxygen followed by nitric acid to afford a mixture of mono- and di-carboxylic acids (pelargonic acid and azelaic acid), which is subsequently treated with different types of solvents for extraction, separation and purification. The product recovery in this method uses water for extraction purpose and is not feasible due to low solubility in water at lower temperatures.

Another patent document GB585315A discloses formation of a mixture of pelargonic acid and azelaic acid in the oxidation of oleic acid using H2SO4, MnO2 and HNO3 wherein the oxidation product is steam distilled. Use of acids and bases results in effluents with dissolved solids and needs further treatment. Using metal oxide catalysts needs extra steps for transforming them into a separable product. The recovery of azelaic acid does not happen from aliphatic ester but needs additional extraction step.

One more patent document U.S. Pat. No. 2,998,439A discloses a process for the separation and recovery of monobasic and dibasic acids e.g. separation of pelargonic acid and azelaic acid, using the steps of counter currently extracting a mixture of said monobasic and dibasic acids between a polar and a non-polar solvent at a temperature above the temperature of dissociation, approximately 85° C., of the acid complexes, the ratio of polar solvent feed rate to acids feed rate being greater than approximately 5, the ratio of non-polar solvent feed rate to acids feed being approximately 1, cooling the polar solution and recovering the dibasic acids contained therein by crystallization, distilling the non-polar solution and separating by distilization the non-polar solvent and the monobasic acids contained therein.

The polar solvent is water and non-polar solvent can be any aliphatic hydrocarbon solvent with a boiling range up to 200° C. This prior art uses water for recovery of azelaic acid and also hydrocarbon solvent with a boiling range up to 200° C. Moreover, it includes temperatures above the dissociation temperature of the products, which actually results in new impurities.

Thus, there is still need in the art to provide a process of recovery and separation of acids conducted at low temperatures, preferably at room temperature. It would be an added industrially advantageous feature of the process when the same provides a pure product in high yields.

### OBJECTIVE OF THE INVENTION

The principal object of the invention is to provide a room-temperature process for the recovery and separation of acids providing the acids in high purity and yield.

### SUMMARY OF THE INVENTION

Accordingly, to accomplish the objects of the invention, a process of recovery and separation of mixtures of mono and di acids comprising:
a) dissolving a mixture of a first acid and a second acid to be recovered and separated in a mixture of water and a first polar solvent in the specific ratio;
b) evaporating the first polar solvent to obtain an aqueous suspension of acids;
c) adding a second polar solvent to the suspension of step (b), wherein boiling point of the first polar solvent is less than the boiling point of the second polar solvent; to obtain separation of water and a solution of one acid in the second solvent and
d) evaporating the second solvent and adding a non-polar solvent, causing precipitation of first or second acid and solution of first or second acid in the non-polar solvent; and
e) filtering the solution of step d) to separate out first precipitated acid from second acid which remains in the non-polar solvent.

In a preferred embodiment, the water and a first polar solvent are mixed in a specific ratio in the range of 5:95 to 15:85.

In a preferred embodiment, the first polar solvent is selected from acetone, isopropyl alcohol, acetonitrile, tetrahydrofuran, methanol, ethanol, dioxane and such like.

In another preferred embodiment, said second polar solvent is selected from ethylene dichloride, or an aliphatic ester, preferably ethyl acetate, butylacetate, propyl acetate and such like.

In yet another preferred embodiment, said non polar solvent is selected from n-hexane, diethylether, butyl ether, pet ether, cyclopentyl methyl ether, n-pentane and such like.

### BRIEF DESCRIPTION OF THE DRAWINGS

FIG. 1 depicts the flow chart of process for recovery and separation of mixtures of mono and di acids.

FIG. 2 depicts ¹H NMR data of azelaic acid in DMSO-d6

FIG. 3 depicts ¹³C NMR data of azelaic acid in DMSO-d6

FIG. 4 depicts ¹H NMR data of pelargonic acid in DMSO-d6

FIG. 5 depicts ¹³C NMR data of pelargonic acid in DMSO-d6

### DETAILED DESCRIPTION OF THE INVENTION

The invention will now be described in detail in connection with certain preferred and optional embodiments, so that various aspects thereof may be more fully understood and appreciated.

In the description of the present invention the terms “Nonanoic acid” and “pelargonic acid” are used synonymously and bear the same meaning throughout the specification. Accordingly, to accomplish the objects of the invention, the present invention provides a process of recovery and separation of mixtures of mono and di acids comprising the steps of:
a) dissolving a mixture of a first acid and a second acid to be recovered and separated in a mixture of water and a first polar solvent in the ratio of 5:95;
b) evaporating the first polar solvent to obtain an aqueous suspension of acids;
c) adding a second polar solvent to the suspension of step (b), wherein the boiling point of the first polar solvent is less than the boiling point of the second polar solvent; to obtain separation of water and a solution of one acid in the second solvent;
d) evaporating the second solvent and adding a non-polar solvent, causing precipitation of first or second acid and solution of first or second acid in the non-polar solvent; and
e) filtering the solution of step d) to separate out first precipitated acid from second acid which remains in the non-polar solvent.

In another embodiment, the water and a first polar solvent are mixed in a specific ratio in the range of 5:95 to 15:85.

Specifically, the water and a first polar solvent are mixed in a specific ratio of 5:95, 8:92, 10:90 and 15:85.

In a preferred embodiment, the first polar solvent is selected from acetone, isopropyl alcohol, acetonitrile, tetrahydrofuran, methanol, ethanol, dioxane and such like.

In another preferred embodiment, said second polar solvent is selected from ethylene dichloride, or an aliphatic ester, preferably ethyl acetate, butylacetate, propyl acetate and such like.

In yet another preferred embodiment, said non polar solvent is selected from n-hexane, diethylether, butyl ether, pet ether, cyclopentyl methyl ether, n-pentane and such like.

Nonanoic acid gets synthesized along with an azelaic acid in oxidation of oleic acid, however the two acids need to be separated and obtained with high levels of purity. The process of the invention fulfills both the objectives of high yield and purity in shorted period of time.

In an embodiment of the present invention, the process is conducted in batch mode or continuous mode.

The step (a) of process is conducted at room temperature (20-30° C.) and step (d) is conducted at a temperature ranging between 5° and 120° C.

The process overcomes the disadvantages of the prior arts processes by being conducted at low temperatures ranging from 20° C. to 50° C., so that no impurities are formed and the acids are obtained with at least 90% purity.

In a preferred embodiment, the first acid and second acids to be recovered and separated are selected from combination of at least two acids such as azelaic acid, pelargonic acid, dodecanoic acid, 3-hydroxyl nonanoic acid, and tridecanedioic acid.

The process provides yield of acids in the range of 80-100% and purity of acids in the range of 90-99.0%.

In an embodiment, batch separation as well as continuous separation is done at room temperature using extraction and decantation method. The continuous separation protocol needs smaller equipment size than the batch process. The separation of both the products is done in pure forms and in a short time and with complete recovery and recycle of solvents. The current process provides pure acids in less than 20 minutes, as against several hours as reported in the prior arts. The average time of prior art processes is around six hours.

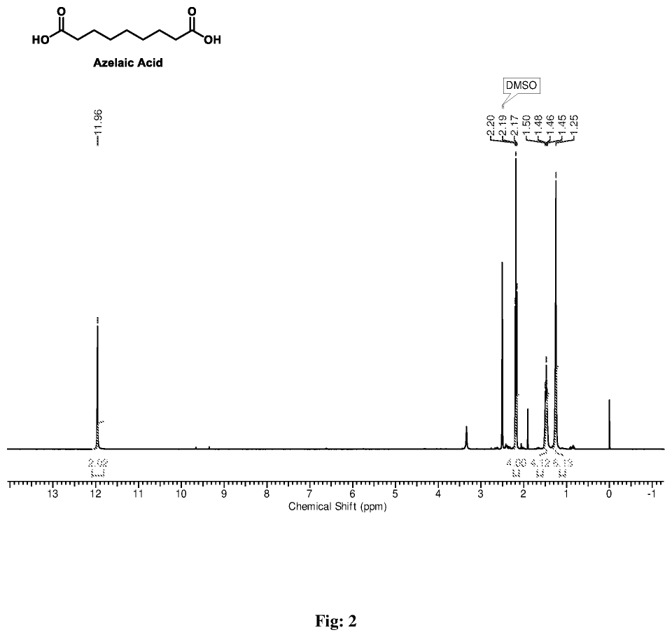

In a preferred embodiment, the continuous separation protocol is 90% more energy efficient than conventional batch operation. Purity of the recovered azelaic acid and pelargonic acid after separation is 90-99% formed without need of any further purification, refer FIGS. 2, 3, 4, and 5.

FIG. 2 depicts ¹H NMR of azelaic acid (400 MHz, DMSO-d6) δ=11.96 (s, 2H), 2.17-2.20 (m, 4H), 1.45-1.50 (t, J=6.4 Hz, 4H), 1.25 (br. s, 6H).

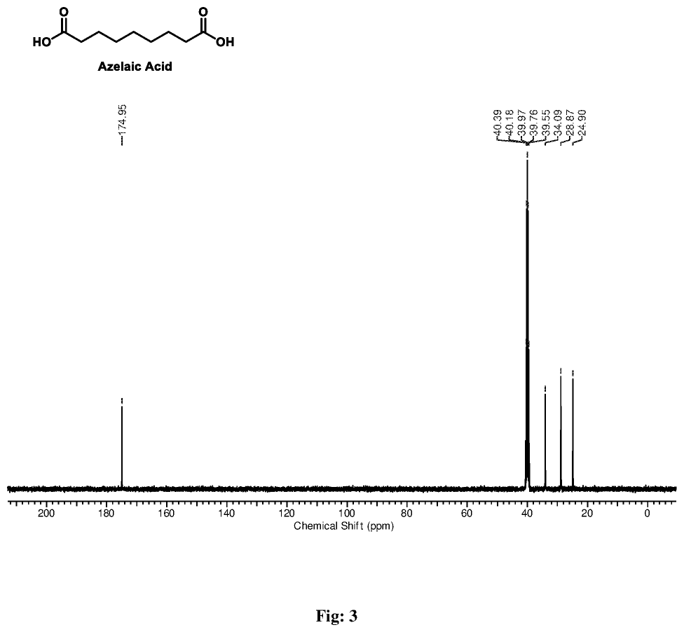

FIG. 3 depicts ¹³C NMR (101 MHz, DMSO-d6) δ=174.9, 34.0, 28.8, 24.9.

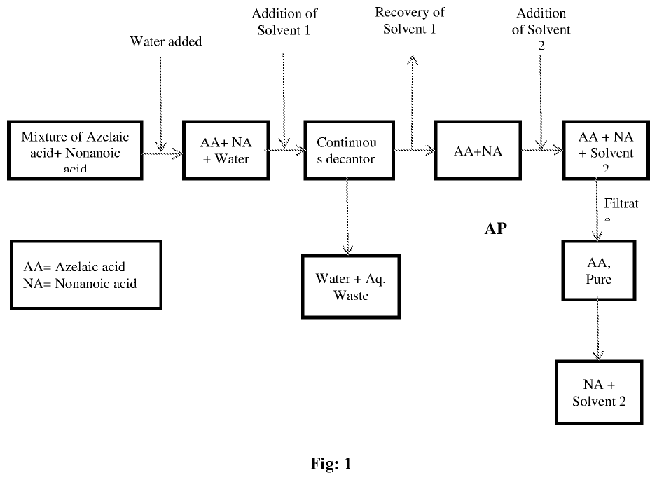

From FIGS. 1 and 2, it is observed that only azelaic acid peaks are observed.

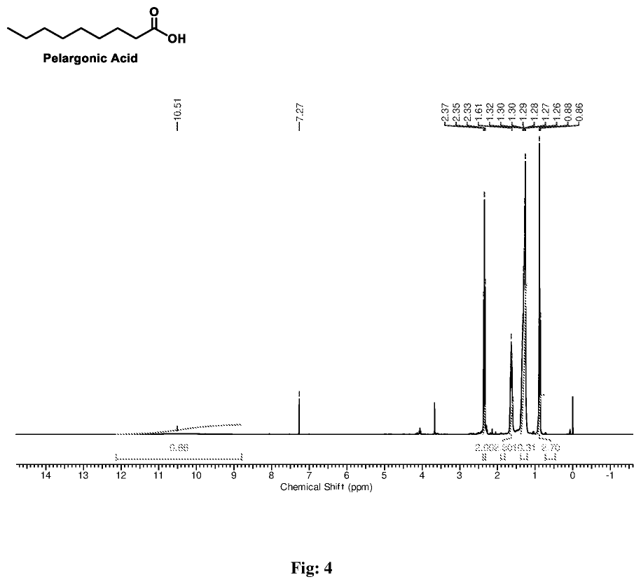

FIG. 4 depicts ¹H NMR of pelargonic acid (400 MHz, DMSO-d6) δ=10.51 (s, 1H), 2.35 (t, J=7.57 Hz, 2H), 1.61 (quin, J=7.38 Hz, 2H), 1.26-1.32 (m, 10H), 0.86-0.88 (m, 3H).

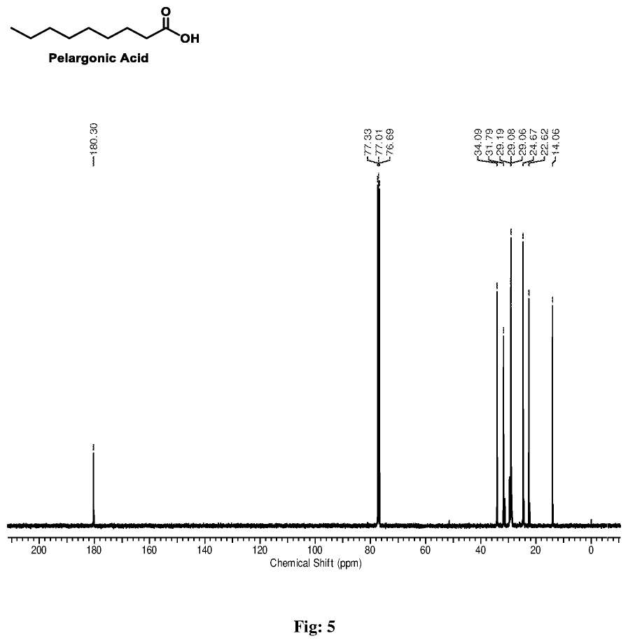

FIG. 5 depicts ¹³C NMR of pelargonic acid (400 MHz, DMSO-d6) δ=180.3, 34.0, 31.7, 29.0, 24.6, 22.6, 14.0.

From FIGS. 4 and 5, it is inferred that only pelargonic acid peaks are present.

FIG. 1 depicts the continuous process for recovery and separation of fatty acids: A mixture of products resulting from the oxidation of oleic acid (with significant portions of azelaic acid and nonanoic acid) and water at ambient conditions are dosed independently using two different pumps respectively to a Tee-mixer followed by a tubular reactor. The outlet of the tubular reactor is connected to another tee-mixer followed by a tubular reactor, where ethyl acetate was pumped independently. The outlet from the tubular reactor was fed to a continuous decanter from where the aqueous layer was decanted and an organic layer comprising of ethyl acetate was continuously fed to recover ethyl acetate and pet ether was added continuously in decanted mass for precipitation of Azelic acid. Precipitated mass was filtered to get the azelaic acid powder and remaining nonanoic acid in the mother liquor.

### EXAMPLES

Following examples are given by way of illustration and therefore should not be construed to limit the scope of the invention.

### Example 1: Comparative Example

2 g mixture of azelaic acid, nonanoic acid in water was taken. Maximum 70% of water could be evaporation under vacuum over 2 hours. To the remaining mass, non-polar solvent i.e. pet ether (60 mL) was added resulting in precipitation of some azelaic acid and nonanoic acid remaining in pet ether solution with inseparable water fraction. Recovered azelaic acid and nonanoic acid had the yield of 61% and 68% respectively. The time required for separation of mixture of acids is 5-10 min and the purity of azelaic acid and nonanoic acid is 98% and 78% respectively.

### Example 2: Comparative Example: This Separation was Done at 0° C.

2 g mixture of azelaic acid and nonanoic acid was taken, to which quantitative amount of water (50 mL) and ethyl acetate (50 mL) were added. Further the aqueous layer was decanted and ethyl acetate was evaporated. Adding non-polar solvent i.e. pet ether (60 mL) to the mixture of azelaic acid and nonanoic acid resulted in precipitation of azelaic acid and nonanoic acid remaining in pet ether solution. Nonanoic acid was recovered quantitatively by evaporating pet ether. The yield of pure azelaic acid and nonanoic acid obtained this way is 84% and 87% respectively.

### Example 3 (Batch Mode): This Separation was Done at 50° C.

2 g mixture of azelaic acid and nonanoic acid was taken, to which quantitative amount of water (50 mL) and ethyl acetate (50 mL) were added. Further the aqueous layer was decanted and ethyl acetate was evaporated. Adding non-polar solvent i.e. pet ether (60 mL) to the mixture of azelaic acid and nonanoic acid resulted in precipitation of azelaic acid and nonanoic acid remaining in pet ether solution. The yield of pure azelaic acid and nonanoic acid obtained this way is 86% and 92% respectively.

### Example 4 (Batch Mode)

Using the mass as given in Example 3, adding 20 ml non-polar solvent i.e. pet ether to the mixture of azelaic acid and nonanoic acid resulted in precipitation of azelaic acid and nonanoic acid remaining in pet ether solution. Nonanoic acid was recovered quantitatively by evaporating pet ether. The yield of pure azelaic acid and nonanoic acid obtained this way is 90% and 86% respectively.

### Example 5 (Batch Mode)

Using the mass as given in Example 3, adding 40 ml non-polar solvent i.e. pet ether to the mixture of azelaic acid and nonanoic acid resulted in precipitation of azelaic acid and nonanoic acid remaining in pet ether solution. The yield of pure azelaic acid and nonanoic acid obtained this way is 88% and 90% respectively.

### Example 6 (Batch Mode)

Using the mass as given in Example 3, adding 80 ml non-polar solvent i.e. pet ether to the mixture of azelaic acid and nonanoic acid resulted in precipitation of azelaic acid and nonanoic acid remaining in pet ether solution. The yield of pure azelaic acid and nonanoic acid obtained this way is 89% and 91% respectively.

### Example 7 (Batch Mode)

Using the mass as given in Example 3, adding 100 ml of non-polar solvent i.e. pet ether to the mixture of azelaic acid and nonanoic acid resulted in precipitation of azelaic acid and nonanoic acid remaining in pet ether solution. The yield of pure azelaic acid and nonanoic acid obtained this way is 84% and 88% respectively.

### Example 8 (Batch Mode)

Using the mass as given in Example 3, adding 120 ml non-polar solvent i.e. pet ether to the mixture of azelaic acid and nonanoic acid resulted in precipitation of azelaic acid and nonanoic acid remaining in pet ether solution. The yield of pure azelaic acid and nonanoic acid obtained this way is 88% and 91% respectively.

### Examples 9 (Continuous Mode)

In the continuous mode of operation, the mixture of acids dissolved in the solvents as given in Example 3 was pumped to a continuous distillation unit for recovering the organic solvent and high boiling residue (mixture of acids and water) that was continuously collected at the bottom of the distillation column was passed to a liquid-liquid extractor for the extraction using polar solvent ethyl acetate (20 volumes when compared to oleic acid). After completion of extraction, the organic phase was separated continuously using a decanter and was subsequently passed to another distillation column where the organic solvent was recovered from the top while the bottom mixture of acids was continuously fed to a tubular reactor along with a non-polar solvent pet-ether (20 volumes when compared to oleic acid) to facilitate precipitation of azelaic acid and nonanoic acid remaining in the solvent. The outlet stream when filtered resulted in azelaic acid with as a main product and in mother liquor the nonanoic acid goes to non-polar solvent. The yield of azelaic acid and nonanoic acid is 89% and 78% respectively. As per above examples 3 to 9, the purity of azelaic acid and nonanoic acid is 98%±0.5% and 78.5%±0.5% respectively.

### Advantages of the Invention

Low temperature process
Zero discharge process
All solvents are recycled
Process provides high yield and purity of products
Time consumption of process is substantially reduced

1-13. (canceled)

14. A process for recovering and separating mixtures of mono acids and di acids, the process comprising:
(a) dissolving a mixture of a first acid and a second acid in a mixture of water and a first polar solvent;
(b) evaporating the first polar solvent from the mixture to obtain an aqueous suspension of the first acid and the second acid;
(c) adding to the aqueous suspension of (b) a second polar solvent having a boiling point greater than the boiling point of the first polar solvent to obtain separation of water and a solution of one of the first acid or the second acid in the second polar solvent;
(d) evaporating the second polar solvent from the solution of (c) and adding a non-polar solvent to the solution, causing a precipitation of the first acid or the second acid and a solution of the first acid or the second acid in the non-polar solvent; and
(e) filtering the solution of (d) to obtain the first acid and the second acid separately.

15. The process according to claim 14, wherein a ratio of water to the first polar solvent in the mixture of (a) is from 5:95 to 15:85.

16. The process according to claim 14, wherein the process is a batch process or a continuous process.

17. The process according to claim 14, wherein (a) is conducted at a temperature from 20° C. to 30° C. and (d) is conducted at a temperature from 50° C. to 120° C.

18. The process according to claim 14, wherein the first acid is azelaic acid.

19. The process according to claim 14, wherein the second acid is selected from the group consisting of nonanoic acid, dodecanoic acid, 3-hydroxyl nonanoic acid, tridecanedioic acid, and mixtures thereof.

20. The process according to claim 14, wherein the first polar solvent is selected from acetone, isopropyl alcohol, acetonitrile, tetrahydrofuran, methanol, ethanol, or dioxane.

21. The process according to claim 14, wherein the second polar solvent is selected from ethylene dichloride, or an aliphatic ester.

22. The process according to claim 14, wherein the second polar solvent is selected from ethylene dichloride, ethyl acetate, butyl acetate, or propyl acetate.

23. The process according to claim 14, wherein the non-polar solvent is selected from n-hexane, diethylether, butyl ether, pet ether, cyclopentyl methyl ether, or n-pentane.

24. The process according to claim 14, wherein the process provides a yield of the first acid from 61% to 90%.

25. The process according to claim 14, wherein the process provides a yield of the second acid from 68% to 92%.

26. The process according to claim 14, wherein a purity of the first acid obtained from the process is from 98% to 99%.

27. The process according to claim 14, wherein a purity of the second acid obtained from the process is from 78% to 79%.
